(19) United States
(12) Patent Application Publication
(10) Pub. No.: US 2016/0230161 A1
(43) Pub. Date: Aug. 11, 2016

(54) PURIFIED LYSIN PROTEIN

(71) Applicant: I-Shou University, Kaohsiung (TW)

(72) Inventors: Chih-Hsin HUNG, Kaohsiung (TW); Huei-Ru LUO, Kaohsiung (TW); Jen-Yi WANG, Kaohsiung (TW)

(21) Appl. No.: 14/700,872

(22) Filed: Apr. 30, 2015

(30) Foreign Application Priority Data
Feb. 10, 2015 (TW) 104104373

(51) Int. Cl. C12N 9/88

(52) U.S. Cl. CPC C12N 9/88; C12Y 402/02002

(57) ABSTRACT

The invention discloses a purified lysin protein for lysing Acinetobacter baumannii cells without pretreatment process using chloroform or EDTA. The purified lysin protein includes a first peptide fragment and a second peptide fragment. The first peptide fragment has an amino acid sequence set forth as SEQ ID NO: 4 and the second peptide fragment has a amino acid sequence set forth as SEQ ID NO: 5.

### BACKGROUND OF THE INVENTION

1. Field of the Invention

The present invention generally relates to a purified protein and, more particularly, to a purified lysin protein.

2. Description of the Related Art

Acinetobacter baumannii, being an opportunistic pathogen, poses ability to survive on artificial surfaces and therefore is frequently detected on hospital settings, such as crash carts, medical apparatuses, tanks or sickbeds. Moreover, A. baumannii strains carrying multi-drug resistance or pan-drug resistance are easily generated, being the important factor of nosocomial infection.

Conventional lysin proteins, also known as endolysins, are hydrolytic enzymes produced by bacteriophages. The bacteriophage infects the host cells (i.e. bacterial cells), produces the conventional lysin protein able to target peptidoglycan, the main component of bacterial cell wall, and able to cleave the bacterial cell wall, leading cell lysis of the host cells. However, A. baumannii is a Gram-negative bacterium with the outer membrane. The presence of the outer membrane prevents the conventional lysin protein from digesting peptidoglycan. And in consequence, a pretreatment process using chloroform or EDTA should be carried out, improving the permeability of the outer membrane of the Gram-negative bacterium, thereby permitting the targeting of the conventional lysin protein to peptidoglycan of the Gram-negative bacterium. Nevertheless, the pretreatment process using chloroform or EDTA resulting in the conventional lysin protein not applicable to not only medical use but also antimicrobial use.

### SUMMARY OF THE INVENTION

It is therefore the objective of this invention to provide a purified lysin protein for lysing A. baumannii cells without the pretreatment process using chloroform or EDTA.

One embodiment of the invention discloses a purified lysin protein including a first peptide fragment and a second peptide fragment. The first peptide fragment has an amino acid sequence as set forth in SEQ ID NO: 4 and the second peptide fragment has an amino acid sequence as set forth in SEQ ID NO: 5.

In a preferred form shown, the purified lysin protein has an amino acid sequence as set forth in SEQ ID NO: 6.

In a preferred form shown, E. coli cells are used for producing the purified lysin protein.

In a preferred form shown, the purified lysin protein is produced by expressing an expression plasmid including a first DNA fragment and a second DNA fragment The first DNA fragment having a nucleic acid sequence as set forth in SEQ ID NO: 1 and the second DNA fragment having a nucleic acid sequence as set forth in SEQ ID NO: 2.

In a preferred form shown, the purified lysin is produced by expressing the expression plasmid having a nucleic acid sequence as set forth in SEQ ID NO: 3.

In a preferred form shown, the purified lysin is produced in E. coli by expressing the expression plasmid deposited at DSMZ-Deutsche Sammlung von Mikroorganismen and Zellkulturen with deposit number DSM 32023.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 depicts a SDS-PAGE demonstrating the production of the purified lysin protein in trial (A).

FIG. 2 depicts images demonstrating the minimum inhibitory concentration of the purified lysin protein in trial (B).

FIG. 3 depicts a bar chart demonstrating the minimum inhibitory concentration of the purified lysin protein in trial (C).

FIG. 4 depicts images demonstrating the thermal tolerance of the purified lysin protein in trial (D).

FIG. 5 depicts images demonstrating the pH tolerance of the purified lysin protein in trial (E).

In the various figures of the drawings, the same numerals designate the same or similar parts. Furthermore, when the term “first”, “second”, “third”, “fourth”, “inner”, “outer” “top”, “bottom” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

A purified lysin protein according to the present invention includes a first peptide fragment and a second peptide fragment. Specifically, the first peptide fragment has an amino acid sequence corresponding to a functional domain of phage T7 tail fiber protein. The purified lysin protein according to the present invention can thus recognize other membrane proteins of A. baumannii cells, specifically targeting A. baumannii cells. Moreover, the second peptide fragment has an amino acid sequence corresponding to a functional domain of pectate lyase superfamily protein, permitting the purified lysin protein according to the present invention lyses polysaccharides on the other membrane of A. baumannii cells and penetrates through the outer membrane of A. baumannii cells. Moreover, the second peptide fragment can also cleave the cell wall of A. baumannii cells, leading cell lysis of the A. baumannii cells.

In general, the purified lysin protein can be produced by any means well-known in the field. For example, the purified lysin protein can be produced by E. coli cells. That is, the expression plasmid is transformed into the E. coli cells, followed by inducing the production of the lysin protein. Subsequently, the purified lysin protein is obtained by purifying the produced lysin protein.

Specifically, the expression plasmid includes a first DNA fragment corresponding to the first peptide fragment and a second DNA fragment corresponding to the second peptide fragment. Moreover, the expression plasmid preferably has the codon usage of E. coli. More detailed, the first DNA fragment has a nucleic acid sequence set forth as SEQ ID NO: 1 and the second DNA fragment has a nucleic acid sequence set forth as SEQ ID NO: 2. Thus, the corresponding first peptide fragment and the corresponding second peptide fragment have amino acid sequences set forth as SEQ ID NO: 4 and 5, respectively.

The construction of the expression plasmid, the induction of the production of the lysin protein, and the purification of the purified lysin protein are the prior art well-known in the field, and therefore are not limited to the following statement. In this embodiment, a DNA fragment with the nucleic acid sequence set forth as SEQ ID NO: 3 constructed from a φkm18p phage is introduced in a pET21b vector to generate the expression plasmid which is deposited at DSMZ-Deutsche Sammlung von Mikroorganismen and Zellkulturen with deposit number DSM 32023. The expression plasmid is then transformed into the E. coli cells (C43(DE3) strain), and the lysin protein with a corresponding amino acid sequence set forth as SEQ ID NO: 6 is produced as follows.

The E. coli cells with the expression plasmid are grown, followed by inducing the production of the lysin protein by IPTG. The produced lysin protein further includes a His tag which can be purified by a Ni²⁺ affinity column to obtain the purified lysin protein according to this embodiment of the present invention.

Accordingly, the purified lysin protein according to this embodiment of the present invention includes the first peptide fragment corresponding to the functional domain of phage T7 tail fiber protein and the second peptide fragment corresponding to the functional domain of pectate lyase superfamily protein, recognizing the outer membrane of A. baumannii cells and penetrating through the outer membrane of A. baumannii cells. Moreover, the purified lysin protein can cleave the cell wall of A. baumannii cells, leading cell lysis of the A. baumannii cells. Therefore, the purified lysin protein can used for lysing A. baumannii cells without the pretreatment process using chloroform or EDTA.

Therefore, the purified lysin protein can be applied in medical use. For instance, the purified lysin protein can be administered to a subject in need thereof to treat for A. baumannii infection. The purified lysin protein can be given individually or combined with any acceptable excipients, for example carriers or other ingredients, and is capable of being further manufactured into any form of medicament, such as pill, capsule, powder, solution, pastil and paste for easy and convenient delivery to the subject in need thereof.

Moreover, the purified lysin protein can also be applied in antimicrobial use. That is, the purified lysin protein can be solved in an appropriate solvent with pH 3-12 to produce a bactericide. The bactericide can be applied in the air by the aerosol spray in 25-50° C. Alternatively, the bactericide can also be used to disinfect the hospital settings by soaking the hospital settings into the bactericide or wiping by the bactericide in 25-50° C.

In order to evaluate the purified lysin protein can be produced, and can be used for lysing A. baumannii cells without the pretreatment process using chloroform or EDTA, the following trials are carried out.

Trial (A). Purification.

The E. coli cells with the expression plasmid (deposit number DSM 32023) are grown, followed by inducing the production of the lysin protein by IPTG. The grown E. coli cells are lysed, and centrifuged to obtain a supernatant and a pellet. The supernatant is mixed with Ni²⁺ resins at 4° C. for 1 hour and loaded in the column. The unbound proteins are washed by a binding buffer containing 50 mM Tris-HCl (pH 8.2), 15 mM MgCl₂, 20% (v/v) glycerol, 0.05% β-ME and 0.1 mM PMSF. Finally, the purified lysin protein is eluted by an elution buffer further including 250 mM imidazole.

Referring to TABLE 1, a cell lysate obtained from the grown E. coli cells before IPTG induction is used as a sample of group A1, a cell lysate obtained from the grown E. coli cells after IPTG induction is used as a sample of group A2, the pellet obtained by centrifugation is used as a sample of group A3, and the purified lysin protein obtained by elution is used as a sample of group A4. The samples of groups A1-A4 are mixed with β-ME and denatured by heating, followed by SDS-PAGE analysis.

TABLE 1

| Groups | IPTG induction | Centrifugation | Elution |
|---|---|---|---|
| A1 | − | − | − |
| A2 | + | − | − |
| A3 | + | + | − |
| A4 | + | + | + |

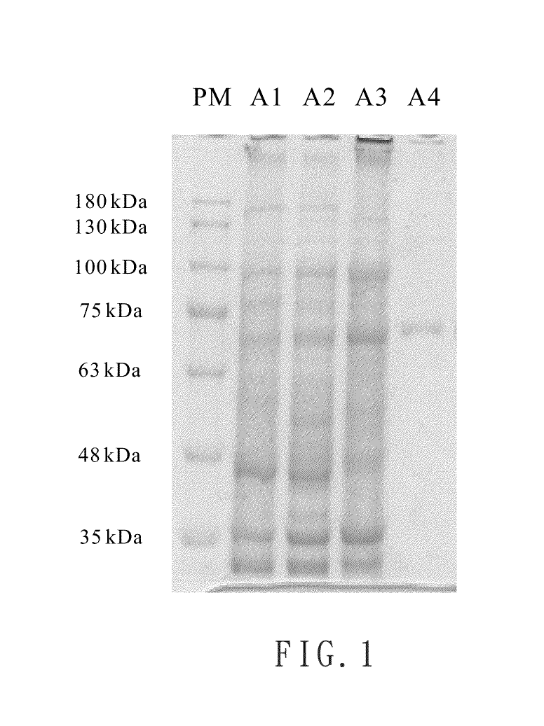

Referring to FIG. 1, compared with the protein marker (PM), all of the samples of groups A2-A4 show a band with molecular weight about 72 kDa, which is similar to the predicted molecular weight of the purified lysin protein.

Trial (B). MIC Test by Paper Disk Diffusion Method.

The minimum inhibitory concentration (MIC) of the purified lysin protein is analyzed by the paper disk diffusion method. The purified lysin proteins of groups B1-B5 with different concentration listed in TABLE 2 are spotted on the paper disks. The paper disks are placed on seeded bacterial lawn (A. baumannii Km18) on the agar surface and incubated for 24 and 72 hours, respectively.

TABLE 2

| Groups | Concentration (μg/20 μL) |
|---|---|
| B1 | 1 |
| B2 | 5 |
| B3 | 10 |
| B4 | 50 |
| B5 | 100 |

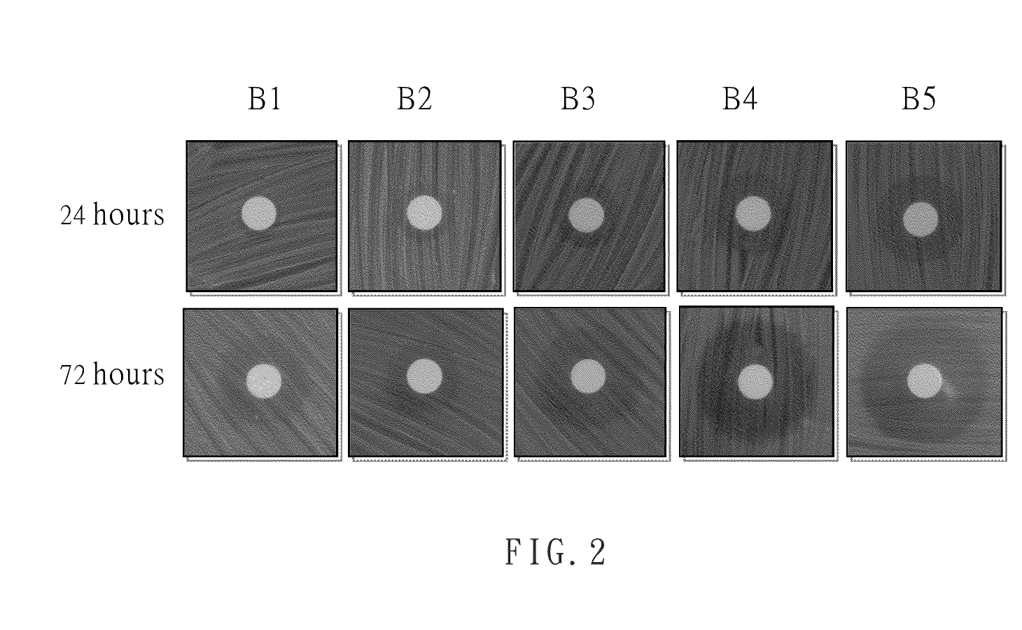

Referring to FIG. 2, after 24-hour incubation, the purified lysin proteins of groups B2-B5 can form inhibition zone on the agar surface. Moreover, after 72-hour incubation, the purified lysin proteins of groups B1-B5 can form inhibition zone on the agar surface.

Trial (C). MIC test in broth medium.

Moreover, overnight cultured A. baumannii Km18 cells are inoculated into fresh LB medium with 50 μg/mL ampicillin and cultured until log phase. Then, the cultured A. baumannii Km18 cells (10⁶ CFU/mL) are mixed with the purified lysin proteins in concentrations listed in TABLE 3, followed by culturing at 37° C. for 6 hours.

TABLE 3

| Groups | Concentration (mg/mL) |
|---|---|
| C0 | 0 |
| C1 | 0.5 |
| C2 | 1.0 |
| C3 | 1.5 |
| C4 | 2.0 |
| C5 | 2.5 |

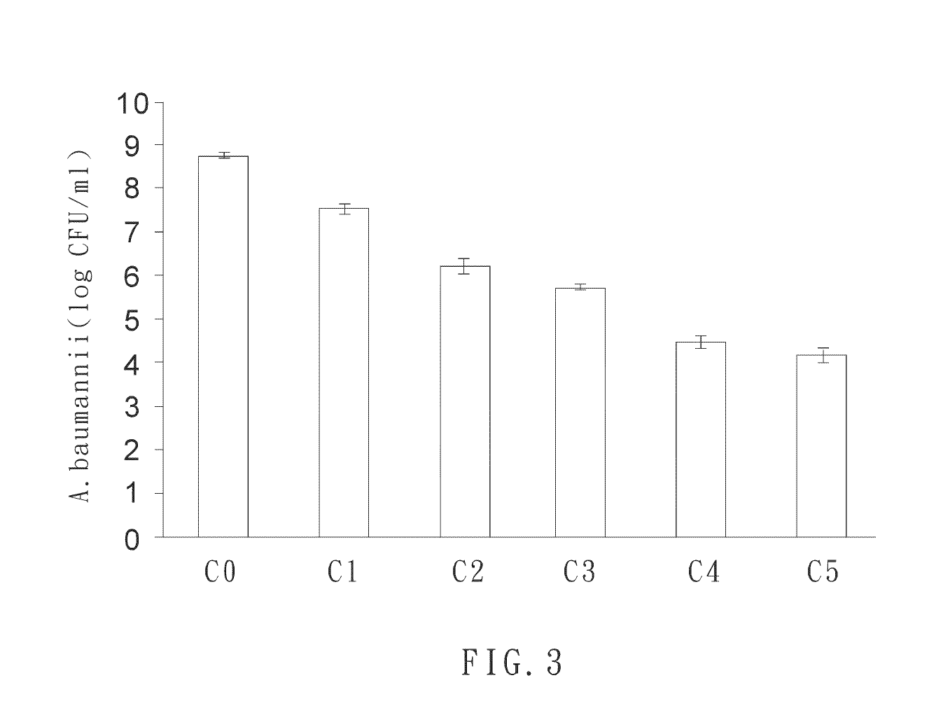

Referring to FIG. 3, after 6-hr culturing, the A. baumannii Km18 cells of group CO reach a concentration of 10⁸ CFU/mL. The purified lysin protein can effectively inhibits the growth of the cultured A. baumannii Km18 cells in a dose-dependent manner, and the cultured A. baumannii Km18 cells of group C5 have a concentration of 10⁴ CFU/mL.

Trial (D). Thermal Tolerance Analysis.

The purified lysin proteins (5 ng) of groups DO-D6 are standed at the temperature listed in TABLE 4 for 30 minutes, respectively. The purified lysin proteins are then analyzed by SDS-PAGE analysis and in-gel plaque formation assay.

TABLE 4

| Groups | Temperature (° C.) |
|---|---|
| D0 | 25 (room temperature) |
| D1 | 40 |
| D2 | 50 |
| D3 | 60 |
| D4 | 70 |
| D5 | 80 |
| D6 | 100 |

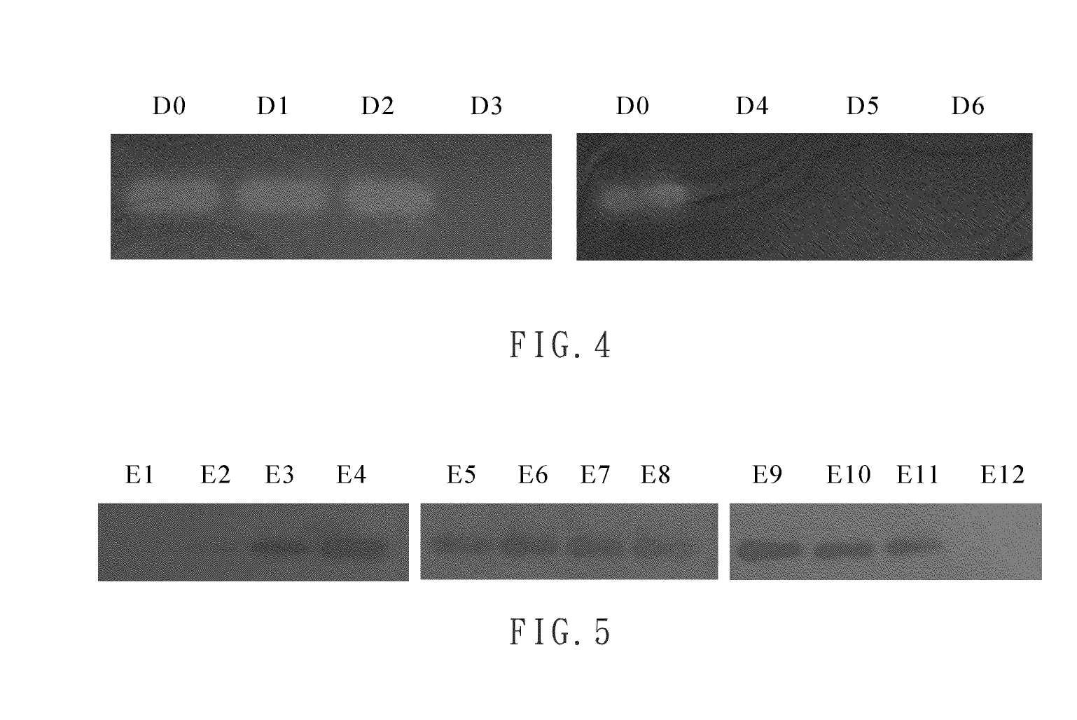

Referring to FIG. 4, the purified lysin proteins of groups D1-D3 have ability to form plaques in-gel. That is, the purified lysin protein is tolerant of environment with temperature of 25-50° C. for 30 minutes.

Trial (E). pH Tolerance Analysis.

Furthermore, the purified lysin proteins (5 ng) of groups E1-E12 are mixed with autoclaved PBS buffer with pH value listed in TABLE 5, and standed at room temperature for 30 minutes, followed by SDS-PAGE analysis and in-gel plaque formation assay.

TABLE 5

| Groups | pH value |
|---|---|
| E1 | 2 |
| E2 | 3 |
| E3 | 4 |
| E4 | 5 |
| E5 | 6 |
| E6 | 7 |
| E7 | 8 |
| E8 | 9 |
| E9 | 10 |
| E10 | 11 |
| E11 | 12 |
| E12 | 13 |

Referring to FIG. 5, the purified lysin proteins of groups E1-E12 have ability to form plaques in-gel. That is, the purified lysin protein is tolerant of environment with pH of 3-12.

Accordingly, the purified lysin protein according to this embodiment of the present invention includes the first peptide fragment corresponding to the functional domain of phage T7 tail fiber protein and the second peptide fragment corresponding to the functional domain of pectate lyase superfamily protein, recognizing the outer membrane of A. baumannii cells and penetrating through the outer membrane of A. baumannii cells. Moreover, the purified lysin protein can cleave the cell wall of A. baumannii cells, leading cell lysis of the A. baumannii cells. Therefore, the purified lysin protein can be applied in medical use to treat for A. baumannii infection.

Moreover, the purified lysin protein according to this embodiment of the present invention can tolerant of environment with temperature of 25-50° C. and pH of 3-12, and therefore is suitable to be used as a bactericide for disinfecting the hospital setting.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A purified lysin protein, comprising: a first peptide fragment and a second peptide fragment, with the first peptide fragment having an amino acid sequence as set forth in SEQ ID NO: 4 and the second peptide fragment having an amino acid sequence as set forth in SEQ ID NO: 5.

2. The purified lysin protein as claimed in claim 1, with the purified lysin protein having an amino acid sequence as set forth in SEQ ID NO: 6.

3. The purified lysin protein as claimed in claim 1, with the purified lysin protein being produced by E. coli cells.

4. The purified lysin protein as claimed in claim 3, with the purified lysin protein being produced by expressing an expression plasmid comprising a first DNA fragment and a second DNA fragment, with the first DNA fragment having a nucleic acid sequence as set forth in SEQ ID NO: 1 and the second DNA fragment having a nucleic acid sequence as set forth in SEQ ID NO: 2.

5. The purified lysin as claimed in claim 4, with the purified lysin being produced by expressing the expression plasmid having a nucleic acid sequence as set forth in SEQ ID NO: 3.

6. The purified lysin as claimed in claim 5, with the purified lysin being produced by expressing the expression plasmid deposited at DSMZ-Deutsche Sammlung von Mikroorganismen and Zelikulturen with deposit number DSM 32023.


SEQUENCE LISTING (6 sequences)

SEQ ID NO: 1 - DNA, 318 bp, Bacteriophage phi-km18p
acacctacagatactttccctatcagttttgaatatgatgagaaatacgatgctgtacatgtctttcttaatgacgtggcagttgaagacttgggatacactgtatcccaagttaatgctgttactctaaaaattgaacctgctattccagaaggtactgttcgtattgaacgtgagactgacattgataagatgaagtacatctttgatgctggtgcgctgtttattgatcagaatgtggatgcggattttagacaaatcgtacattcccagcaagaggtacgtgatggttttattaaactacgtggtgatgtacta

SEQ ID NO: 2 - DNA, 720 bp, Bacteriophage phi-km18p
ctaactgttgagaattttggtgctaaaggtgatggggttacagacgatagcgcagcattccaagcatattgtgatagcccatttacaggtgctaatattcgtctagctaatcgtagagctgtgtatatcattaaaaagcaagtggactgtaagggtaagggtattgtaggtaatggttttggtaaacaatctcaagctgcttatgacctaagcagtatccgtgtaatggaaggggactacacgaacagtaatgccgatctagccgatattgcatttattaacgttggtgcggaagtccgtaacctacaattcgtatctagtaatctaggtactattagtggtattaatgttagtggttataatacaacaatcaataatgtgaacttcacaggttttaagaatcaggtgcatgtagtaggtgcttccgtacgtttcagtgtgtctgatctaacatctattggtgcagcaaatgctgggttttatttccgtgataaacagagcgatcaaagtacaactgcgtacttcaatcagtgctcatggcagtggggtaataaagctgtagtctttgctaaagaagcatatggttgtgttttccgtgataacatcgtcgagtatatggatggtggttttgaggcttctgtattctctaactgtatatttgaaggtaactggtgtgaatcaactcgtagtggtaatgctgttgattggattgtcaacact

SEQ ID NO: 3 - DNA, 2082 bp, Bacteriophage phi-km18p
atgaatatactacgctcatttacagagacagtggtgactacacctacagatactttccctatcagttttgaatatgatgagaaatacgatgctgtacatgtctttcttaatgacgtggcagttgaagacttgggatacactgtatcccaagttaatgctgttactctaaaaattgaacctgctattccagaaggtactgttcgtattgaacgtgagactgacattgataagatgaagtacatctttgatgctggtgcgctgtttattgatcagaatgtggatgcggattttagacaaatcgtacattcccagcaagaggtacgtgatggttttattaaactacgtggtgatgtactaccattagtacatggtttacaagaagctttgcaacaagcacaggaggctagcgaagctgctcaagaagctgctgctgctgcggaagaggctgctcagcaaacacgaatggctgagaaggttattgataaatctggactgactcaacaacaaatcaacgatgcttggctaactgttgagaattttggtgctaaaggtgatggggttacagacgatagcgcagcattccaagcatattgtgatagcccatttacaggtgctaatattcgtctagctaatcgtagagctgtgtatatcattaaaaagcaagtggactgtaagggtaagggtattgtaggtaatggttttggtaaacaatctcaagctgcttatgacctaagcagtatccgtgtaatggaaggggactacacgaacagtaatgccgatctagccgatattgcatttattaacgttggtgcggaagtccgtaacctacaattcgtatctagtaatctaggtactattagtggtattaatgttagtggttataatacaacaatcaataatgtgaacttcacaggttttaagaatcaggtgcatgtagtaggtgcttccgtacgtttcagtgtgtctgatctaacatctattggtgcagcaaatgctgggttttatttccgtgataaacagagcgatcaaagtacaactgcgtacttcaatcagtgctcatggcagtggggtaataaagctgtagtctttgctaaagaagcatatggttgtgttttccgtgataacatcgtcgagtatatggatggtggttttgaggcttctgtattctctaactgtatatttgaaggtaactggtgtgaatcaactcgtagtggtaatgctgttgattggattgtcaacactagccaccagcagttatttaactgtaaattcggagtcaactacatccgtgccccgtggttagatcgtactaaccctaacgatattgctggttctaacaatgctggtggtattcaggcgaagaacagtgctgtcgcagtaacaggtgctacaggggcaaaaatcagattaaatacgaatggtttaagtactggttttgctgattggtttggtgtttcaaacagtccattaagtagtcgcgcactactgcttacaacacaagaccgagcatctggaagtaatttcgatacacctatcgttattgctgctccaaacggttgcctgtgggaaagaaataggagcgctacagattatacaccagtaacaaaacgaagattaattggagctaatgcggataatactgctgcatactatggggttgatacttatactaagcgtgtacgtaagtggagtacatttgatcacaccactaatacctctggtcaattcttagcacctattatgctaacttatgattctgctgcaactactcagcaagttaatgcaggttggagtattactaaggaagcaggtactacaggtatttatattttacaaagaactgcatcaactgtagcgcctatggctaaccctaattttgtagttgggggtatattcactggttctgctacaggtactctcgctgcggttagttatagtctacaggctattgagacttactcagggtcttggacagtgtataaagaggctgctggatttaagatagcttttagagatcagactggtgcattagtcaatgttaacagattcaccgctatgtttactgtggtatctggattttaa

SEQ ID NO: 4 - protein, 106 aa, Bacteriophage phi-km18p
TPTDTFPISFEYDEKYDAVHVFLNDVAVEDLGYTVSQVNAVTLKIEPAIPEGTVRIERETDIDKMKYIFDAGALFIDQNVDADFRQIVHSQQEVRDGFIKLRGDVL

SEQ ID NO: 5 - protein, 240 aa, Bacteriophage phi-km18p
LTVENFGAKGDGVTDDSAAFQAYCDSPFTGANIRLANRRAVYIIKKQVDCKGKGIVGNGFGKQSQAAYDLSSIRVMEGDYTNSNADLADIAFINVGAEVRNLQFVSSNLGTISGINVSGYNTTINNVNFTGFKNQVHVVGASVRFSVSDLTSIGAANAGFYFRDKQSDQSTTAYFNQCSWQWGNKAVVFAKEAYGCVFRDNIVEYMDGGFEASVFSNCIFEGNWCESTRSGNAVDWIVNT

SEQ ID NO: 6 - protein, 693 aa, Bacteriophage phi-km18p
MNILRSFTETVVTTPTDTFPISFEYDEKYDAVHVFLNDVAVEDLGYTVSQVNAVTLKIEPAIPEGTVRIERETDIDKMKYIFDAGALFIDQNVDADFRQIVHSQQEVRDGFIKLRGDVLPLVHGLQEALQQAQEASEAAQEAAAAAEEAAQQTRMAEKVIDKSGLTQQQINDAWLTVENFGAKGDGVTDDSAAFQAYCDSPFTGANIRLANRRAVYIIKKQVDCKGKGIVGNGFGKQSQAAYDLSSIRVMEGDYTNSNADLADIAFINVGAEVRNLQFVSSNLGTISGINVSGYNTTINNVNFTGFKNQVHVVGASVRFSVSDLTSIGAANAGFYFRDKQSDQSTTAYFNQCSWQWGNKAVVFAKEAYGCVFRDNIVEYMDGGFEASVFSNCIFEGNWCESTRSGNAVDWIVNTSHQQLFNCKFGVNYIRAPWLDRTNPNDIAGSNNAGGIQAKNSAVAVTGATGAKIRLNTNGLSTGFADWFGVSNSPLSSRALLLTTQDRASGSNFDTPIVIAAPNGCLWERNRSATDYTPVTKRRLIGANADNTAAYYGVDTYTKRVRKWSTFDHTTNTSGQFLAPIMLTYDSAATTQQVNAGWSITKEAGTTGIYILQRTASTVAPMANPNFVVGGIFTGSATGTLAAVSYSLQAIETYSGSWTVYKEAAGFKIAFRDQTGALVNVNRFTAMFTVVSGF